SEQUENCE LISTING (6 sequences)

SEQ ID NO: 1 - DNA, 50 bp, Artificial Sequence
  Ultra High-throughput Test Fragment 2 (UHTF2)
tacgcaagctaagccatatccttattaaagcttttttgaaagttaatcgt

SEQ ID NO: 2 - DNA, 50 bp, Artificial Sequence
  Ultra High-throughput Test Fragment 79 (UHTF 79)
atcgttcccccgcggcgccgggcccggcactaggcgggggcctaggtgca

SEQ ID NO: 3 - DNA, 50 bp, Artificial Sequence
  Ultra High-throughput Test Fragment 90 (UHTF 90)
acgtaaaaacttaaaactcaaactcaacgcaccagcctatgcgcctggtc

SEQ ID NO: 4 - DNA, 50 bp, Artificial Sequence
  Ultra High-throughput Test Fragment 100 (UHTF 100)
gcatccgagccctatcccctatccccctattctcttgaggagagtcaacc

SEQ ID NO: 5 - DNA, 50 bp, Artificial Sequence
  Ultra High-throughput Test Fragment 120 (UHTF 120)
gtacggatagggataggggatcgggggataggcttaactcatttctctac

SEQ ID NO: 6 - DNA, 50 bp, Artificial Sequence
  Ultra High-throughput Test Fragment 150 (UHTF 150)
cgtatttttacgttttagatttagattgcgttataacccatactaggggg

(19) United States
(12) Patent Application Publication
(10) Pub. No.: US 2013/0217023 A1
(43) Pub. Date: Aug. 22, 2013

(54) System And Method For Generation And Use Of Compact Clonally Amplified Products

(71) Applicant: 454 Life Sciences Corporation, (US)

(72) Inventors: Brian Christopher Godwin, North Haven, CT (US); Priya Shanbhag, Rocky Hill, CT (US); Craig Elder Mealmaker, New Haven, CT (US)

(73) Assignee: 454 LIFE SCIENCES CORPORATION, Branford, CT (US)

(21) Appl. No.: 13/765,223

(22) Filed: Feb. 12, 2013

Related U.S. Application Data
(60) Provisional application No. 61/601,824, filed on Feb. 22, 2012.

(51) Int. Cl. C12Q 1/68

(52) U.S. Cl. CPC C12Q 1/6876
USPC 435/6.12; 536/24.33

(57) ABSTRACT

A method for sequencing a nucleic acid is described that comprises the steps of: coupling an adaptor to at least one end of a template nucleic acid molecule; circularizing the adaptor coupled nucleic acid molecule; amplifying the adaptor coupled nucleic acid molecule to form a linear amplified concatamer molecule comprising a plurality of copies of the template nucleic acid molecule; compacting the linear amplified concatamer molecule with a branched polyelectrolyte species to form a branched polyelectrolyte compacted amplified concatamer molecule; and sequencing the branched polyelectrolyte compacted amplified concatamer molecule to produce a sequence composition of the template nucleic acid molecule.

### RELATED APPLICATIONS

This application claims priority to U.S. Ser. No. 61/601,824, filed Feb. 22, 2012, the contents of which are herein incorporated by reference in their entirety.

### FIELD OF THE INVENTION

The invention provides methods, reagents and kits for producing a compacted, clonally amplified nucleic acid concatamer product.

### INCORPORATION BY REFERENCE OF SEQUENCE LISTING

The contents of the text file named 21465_{—}552001US_ST25.txt”, which was created on Jan. 31, 2013 and is 2 KB in size, are hereby incorporated by reference in their entirety.

### BACKGROUND OF THE INVENTION

Compact structures of nucleic acid molecules that comprise a sequence composition of concatenated copies of a template sequence are sometimes referred to as a “nanoball” or “rolony”, and have been used as templates in a number of nucleic acid sequencing technologies. The use of concatenated template molecule embodiments in sequencing applications provide advantages that include, but are not limited to, the fact that they form a compact structure and are amendable to immobilization onto a solid phase substrate. A high copy number of template sequence in the concatenated molecule provides for signal amplification that, when immobilized remains stationary during processing and requires very little space (i.e. typically on the nanometer scale).

In typical embodiments, the concatamers of amplified copies can be generated using methods referred to as rolling circle amplification (sometimes referred to as “RCA”), or rolling circle replication (sometimes referred to as RCR). Rolling circle techniques are well known to those of ordinary skill who appreciate that they are efficient methods to amplify/replicate a circular template nucleic acid to produce long (>10 kb) single stranded linear DNA molecules that comprise concatenated copies of the template nucleic acid sequence composition. Examples of rolling circle methods useful for producing linear concatamer molecules are described in Blanco et al. (Blanco et al., Highly Efficient DNA Synthesis by the Phage φ29 DNA Polymerase; J. Biol. Chem. 264, 8935 (1989)); and Drmanac et al. (Drmanac et al., Human Genome Sequencing Using Unchained Base Reads on Self-Assembling DNA Nanoarrays; Science 327(5961):78-81 (2009)), both of which are hereby incorporated by reference herein in its entirety for all purposes.

In typical embodiments, a number of amplified or replicated copies of a nucleic acid template molecule are generated (the number of copies typically depends on how many cycles of RCA/RCR amplification are employed but includes numbers from 100's of copies or 1,000's of copies) to form a single stranded nucleic acid molecule comprising a concatamer of the amplified copies useful for signal amplification in sequencing process'. Further, the concatamerized nucleic acid molecule preferably comprises sequence composition, at least in portions of the molecule, which promote the formation of secondary structure and induces the molecule to fold creating a measure of compaction that is typically dependent on the sequence composition. For example, in a typical sequencing application an adaptor sequence designed to promote secondary structure formation is ligated to at least one end of a nucleic acid template to be sequenced prior to the RCA process which results in a concatamerized molecule with repeating regions that promote folding of the molecule.

However, in many instances the sequence composition of the molecule alone is insufficient to promote strong binding in the secondary structure resulting in subsequent dissolution of the secondary structure when subjected to further processing (i.e. as in a sequencing process) linearizing the molecule that reduces the desired compaction. In the typical sequencing applications described above this puts a functional limit on the length of nucleic acid template to be sequenced. For example, particularly for nucleic acid templates to be sequenced that do not comprise sequence composition that promotes secondary structure formation, as the length increases the degree of initial compaction decreases and probability of dissolution increases. Also in the present example, the length of the adaptor sequence can be increased to promote formation of a stable secondary structure, and in many instances to lengths of these adaptor sequences are longer than the length of the nucleic acid template to be sequenced.

The presently described invention enables dramatic increases in the length of the nucleic acid template to be sequenced relative to the length of an adaptor and further reduces the reliance on the sequence composition of the adaptor to promote secondary structure formation.

### SUMMARY OF THE INVENTION

Embodiments of the invention relate to the determination of the sequence of nucleic acids. More particularly, embodiments of the invention relate to methods and systems for producing and sequencing a compacted, clonally amplified nucleic acid concatamer product.

An embodiment of a method for sequencing a nucleic acid is described that comprises the steps of: coupling an adaptor to at least on end of a template nucleic acid molecule; circularizing the adaptor coupled nucleic acid molecule; amplifying the adaptor coupled nucleic acid molecule to form a linear amplified concatamer molecule comprising a plurality of copies of the template nucleic acid molecule; compacting the linear amplified concatamer molecule with a branched polyelectrolyte species to form a branched polyelectrolyte compacted amplified concatamer molecule; and sequencing the branched polyelectrolyte compacted amplified concatamer molecule to produce a sequence composition of the template nucleic acid molecule.

Also, an embodiment of a nucleic acid molecule is described that comprises: a compacted nucleic acid concatamer that includes a plurality of copies of a template nucleic acid sequence, a plurality of copies of an adaptor sequence, and one or more branched polyelectrolyte molecules operatively coupled to a backbone of the compacted nucleic acid concatamer.

Further, an embodiment of a method for sequencing a nucleic acid, comprising the steps of: performing rolling circle amplification of a nucleic acid template in the presence of a branched polyelectrolyte species to form a branched polyelectrolyte compacted amplified concatamer of the nucleic acid template; and sequencing the branched polyelectrolyte compacted amplified concatamer molecule to produce a sequence composition of the nucleic acid template.

The above embodiments and implementations are not necessarily inclusive or exclusive of each other and may be combined in any manner that is non-conflicting and otherwise possible, whether they be presented in association with a same, or a different, embodiment or implementation. The description of one embodiment or implementation is not intended to be limiting with respect to other embodiments and/or implementations. Also, any one or more function, step, operation, or technique described elsewhere in this specification may, in alternative implementations, be combined with any one or more function, step, operation, or technique described in the summary. Thus, the above embodiment and implementations are illustrative rather than limiting.

### BRIEF DESCRIPTION OF THE DRAWINGS

The above and further features will be more clearly appreciated from the following detailed description when taken in conjunction with the accompanying drawings. In the drawings, like reference numerals indicate like structures, elements, or method steps and the leftmost digit of a reference numeral indicates the number of the figure in which the references element first appears (for example, element 160 appears first in FIG. 1). All of these conventions, however, are intended to be typical or illustrative, rather than limiting.

FIG. 1 is a functional block diagram of one embodiment of a sequencing instrument under computer control and a reaction substrate;

FIGS. 2A-2D are a simplified graphical examples of an embodiment depicting stages of generating a compacted amplified concatamer molecule;

FIG. 3 is a simplified graphical example of one embodiment of a dendrimer molecule;

FIG. 4 is a graphical example of one embodiment of amplified concatamer molecules generated from multiple species of template nucleic acid molecule and subjected to a single cycle of sequencing by synthesis;

FIG. 5 is a graphical example of one embodiment of a comparison of amplified concatamer molecules generated from a single species of template nucleic acid molecule with different concentration of dendrimer molecules added during amplification and subjected to a single cycle of sequencing by synthesis;

FIG. 6 is a graphical example of one embodiment of a comparison of amplified concatamer molecules generated from a single species of template nucleic acid molecule with different concentration of dendrimer molecules added after amplification and subjected to a single cycle of sequencing by synthesis;

FIG. 7 is a graphical example of one embodiment of a comparison of amplified concatamer molecules generated from multiple species of template nucleic acid molecule with and without a concentration of dendrimer molecules and subjected to a single cycle of sequencing by synthesis;

FIG. 8 is a graphical example of one embodiment of a comparison of amplified concatamer molecules generated from multiple species of template nucleic acid molecule with and without a concentration of dendrimer molecules and subjected to a 13 cycles of sequencing by synthesis;

FIG. 9 is a graphical example of one embodiment of a comparison of amplified concatamer molecules generated from multiple species of template nucleic acid molecule with and without a concentration of dendrimer molecules and subjected to 25 cycles of sequencing by synthesis;

FIG. 10 is a graphical example of one embodiment of detected signals from amplified concatamer molecules generated from a single species of template nucleic acid without dendrimers over 50 cycles of sequencing by synthesis;

FIG. 11 is a graphical example of one embodiment of detected signals from amplified concatamer molecules generated from a single species of template nucleic acid with dendrimers over 50 cycles of sequencing by synthesis;

FIGS. 12A and 12B are a graphical examples of embodiments of comparisons of the average of mean signal intensity in n=2 fields of view when using G6 and G7 dendrimers respectively at specified concentrations; and

FIG. 13 is a graphical example of one embodiment of a comparison of detected pH changes from amplified concatamer molecules generated from a single species of template nucleic acid molecule with and without a concentration of dendrimer molecules and subjected to incorporation of a single nucleotide species.

### DETAILED DESCRIPTION OF THE INVENTION

As will be described in greater detail below, embodiments of the presently described invention include methods, reagents and kits for producing a stable compacted structure of a single stranded concatamer of clonally amplified nucleic acid template that is resistant to dissolution in sequencing applications.

### a. General

The term “flowgram” generally refers to a graphical representation of sequence data generated by SBS methods, particularly pyrophosphate based sequencing methods (also referred to as “pyrosequencing”) and may be referred to more specifically as a “pyrogram”.

The term “read” or “sequence read” as used herein generally refers to the entire sequence data obtained from a single nucleic acid template molecule or a population of a plurality of substantially identical copies of the template nucleic acid molecule.

The terms “run” or “sequencing run” as used herein generally refer to a series of sequencing reactions performed in a sequencing operation of one or more template nucleic acid molecules.

The term “flow” as used herein generally refers to a single cycle that is typically part of an iterative process of introduction of fluid solution to a reaction environment comprising a template nucleic acid molecule, where the solution may include a nucleotide species for addition to a nascent molecule or other reagent, such as buffers, wash solutions, or enzymes that may be employed in a sequencing process or to reduce carryover or noise effects from previous flows of nucleotide species.

The term “flow cycle” as used herein generally refers to a sequential series of flows where a fluid comprising a nucleotide species is flowed once during the cycle (i.e. a flow cycle may include a sequential addition in the order of T, A, C, G nucleotide species, although other sequence combinations are also considered part of the definition). In some embodiments, the flow cycle is a repeating cycle having the same sequence of flows from cycle to cycle. In other embodiments, the flow cycle may include a non-repeating order of flows which are different from cycle to cycle.

The term “read length” as used herein generally refers to an upper limit of the length of a template molecule that may be reliably sequenced. There are numerous factors that contribute to the read length of a system and/or process including, but not limited to the degree of GC content in a template nucleic acid molecule.

The term “signal droop” as used herein generally refers to a decline in detected signal intensity as read length increases.

The term “test fragment” or “TF” as used herein generally refers to a nucleic acid element of known sequence composition that may be employed for quality control, calibration, or other related purposes.

The term “primer” as used herein generally refers to an oligonucleotide that acts as a point of initiation of DNA synthesis under conditions in which synthesis of a primer extension product complementary to a nucleic acid strand is induced in an appropriate buffer at a suitable temperature. A primer is preferably a single stranded oligodeoxyribonucleotide.

A “nascent molecule” generally refers to a DNA strand which is being extended by the template-dependent DNA polymerase by incorporation of nucleotide species which are complementary to the corresponding nucleotide species in the template molecule.

The terms “template nucleic acid”, “template molecule”, “target nucleic acid”, or “target molecule” generally refer to a nucleic acid molecule that is the subject of a sequencing reaction from which sequence data or information is generated.

The term “nucleotide species” as used herein generally refers to the identity of a nucleic acid monomer including purines (Adenine, Guanine) and pyrimidines (Cytosine, Uracil, Thymine) typically incorporated into a nascent nucleic acid molecule. “Natural” nucleotide species include, e.g., adenine, guanine, cytosine, uracil, and thymine. Modified versions of the above natural nucleotide species include, without limitation, hypoxanthine, xanthine, 7-methylguanine, 5,6-dihydrouracil, and 5-methylcytosine.

The term “monomer repeat” or “homopolymers” as used herein generally refers to two or more sequence positions comprising the same nucleotide species (i.e. a repeated nucleotide species).

The term “homogeneous extension” as used herein generally refers to the relationship or phase of an extension reaction where each member of a population of substantially identical template molecules is homogenously performing the same extension step in the reaction.

The term “completion efficiency” as used herein generally refers to the percentage of nascent molecules that are properly extended during a given flow.

The term “incomplete extension rate” as used herein generally refers to the ratio of the number of nascent molecules that fail to be properly extended over the number of all nascent molecules.

The term “genomic library” or “shotgun library” as used herein generally refers to a collection of molecules derived from and/or representing an entire genome (i.e. all regions of a genome) of an organism or individual.

The term “amplicon” as used herein generally refers to selected amplification products, such as those produced from Polymerase Chain Reaction or Ligase Chain Reaction techniques.

The term “variant” or “allele” as used herein generally refers to one of a plurality of species each encoding a similar sequence composition, but with a degree of distinction from each other. The distinction may include any type of variation known to those of ordinary skill in the related art, that include, but are not limited to, polymorphisms such as single nucleotide polymorphisms (SNPs), insertions or deletions (the combination of insertion/deletion events are also referred to as “indels”), differences in the number of repeated sequences (also referred to as tandem repeats), and structural variations.

The term “allele frequency” or “allelic frequency” as used herein generally refers to the proportion of all variants in a population that is comprised of a particular variant.

The term “key sequence” or “key element” as used herein generally refers to a nucleic acid sequence element (typically of about 4 sequence positions, i.e., TGAC or other combination of nucleotide species) associated with a template nucleic acid molecule in a known location (i.e., typically included in a ligated adaptor element) comprising known sequence composition that is employed as a quality control reference for sequence data generated from template molecules. The sequence data passes the quality control if it includes the known sequence composition associated with a Key element in the correct location.

The term “keypass” or “keypass well” as used herein generally refers to the sequencing of a full length nucleic acid test sequence of known sequence composition (i.e., a “test fragment” or “TF” as referred to above) in a reaction well, where the accuracy of the sequence derived from TF sequence and/or Key sequence associated with the TF or in an adaptor associated with a target nucleic acid is compared to the known sequence composition of the TF and/or Key and used to measure of the accuracy of the sequencing and for quality control. In typical embodiments, a proportion of the total number of wells in a sequencing run will be keypass wells which may, in some embodiments, be regionally distributed.

The term “blunt end” as used herein is interpreted consistently with the understanding of one of ordinary skill in the related art, and generally refers to a linear double stranded nucleic acid molecule having an end that terminates with a pair of complementary nucleotide base species, where a pair of blunt ends are typically compatible for ligation to each other.

The term “sticky end” or “overhang” as used herein is interpreted consistently with the understanding of one of ordinary skill in the related art, and generally refers to a linear double stranded nucleic acid molecule having one or more unpaired nucleotide species at the end of one strand of the molecule, where the unpaired nucleotide species may exist on either strand and include a single base position or a plurality of base positions (also sometimes referred to as “cohesive end”).

The term “SPRI” as used herein is interpreted consistently with the understanding of one of ordinary skill in the related art, and generally refers to the patented technology of “Solid Phase Reversible Immobilization” wherein target nucleic acids are selectively precipitated under specific buffer conditions in the presence of beads, where said beads are often carboxylated and paramagnetic. The precipitated target nucleic acids immobilize to said beads and remain bound until removed by an elution buffer according to the operator's needs (DeAngelis, Margaret M. et al: Solid-Phase Reversible Immobilization for the Isolation of PCR Products. Nucleic Acids Res (1995), Vol. 23:22; 4742-4743, which is hereby incorporated by reference herein in its entirety for all purposes).

The term “carboxylated” as used herein is interpreted consistently with the understanding of one of ordinary skill in the related art, and generally refers to the modification of a material, such as a microparticle, by the addition of at least one carboxyl group. A carboxyl group is either COOH or COO—.

The term “paramagnetic” as used herein is interpreted consistently with the understanding of one of ordinary skill in the related art, and generally refers to the characteristic of a material wherein said material's magnetism occurs only in the presence of an external, applied magnetic field and does not retain any of the magnetization once the external, applied magnetic field is removed.

The term “bead” or “bead substrate” as used herein generally refers to any type of solid phase particle of any convenient size, of irregular or regular shape and which is fabricated from any number of known materials such as cellulose, cellulose derivatives, acrylic resins, glass, silica gels, polystyrene, gelatin, polyvinyl pyrrolidone, co-polymers of vinyl and acrylamide, polystyrene cross-linked with divinylbenzene or the like (as described, e.g., in Merrifield, Biochemistry 1964, 3, 1385-1390), polyacrylamides, latex gels, polystyrene, dextran, rubber, silicon, plastics, nitrocellulose, natural sponges, silica gels, control pore glass, metals, cross-linked dextrans (e.g., Sephadex™) agarose gel (Sepharose™), and other solid phase bead supports known to those of skill in the art.

The term “reaction environment” as used herein generally refers to a volume of space in which a reaction can take place typically where reactants are at least temporarily contained or confined allowing for detection of at least one reaction product. Examples of a reaction environment include but are not limited to cuvettes, tubes, bottles, as well as one or more depressions, wells, or chambers on a planar or non-planar substrate.

The term “virtual terminator” as used herein generally refers to terminators substantially slow reaction kinetics where additional steps may be employed to stop the reaction such as the removal of reactants.

Some exemplary embodiments of systems and methods associated with sample preparation and processing, generation of sequence data, and analysis of sequence data are generally described below, some or all of which are amenable for use with embodiments of the presently described invention. In particular, the exemplary embodiments of systems and methods for preparation of template nucleic acid molecules, amplification of template molecules, generating target specific amplicons and/or genomic libraries, sequencing methods and instrumentation, and computer systems are described.

In typical embodiments, the nucleic acid molecules derived from an experimental or diagnostic sample should be prepared and processed from its raw form into template molecules amenable for high throughput sequencing. The processing methods may vary from application to application, resulting in template molecules comprising various characteristics. For example, in some embodiments of high throughput sequencing, it is preferable to generate template molecules with a sequence or read length that is at least comparable to the length that a particular sequencing method can accurately produce sequence data for. In the present example, the length may include a range of about 25-30 bases, about 50-100 bases, about 200-300 bases, about 350-500 bases, about 500-1000 bases, greater than 1000 bases, or any other length amenable for a particular sequencing application. In some embodiments, nucleic acids from a sample, such as a genomic sample, are fragmented using a number of methods known to those of ordinary skill in the art. In preferred embodiments, methods that randomly fragment (i.e. do not select for specific sequences or regions) nucleic acids and may include what is referred to as nebulization or sonication methods. It will, however, be appreciated that other methods of fragmentation, such as digestion using restriction endonucleases, may be employed for fragmentation purposes. Also in the present example, some processing methods may employ size selection methods known in the art to selectively isolate nucleic acid fragments of the desired length.

Also, it is preferable in some embodiments to associate additional functional elements with each template nucleic acid molecule. The elements may be employed for a variety of functions including, but not limited to, primer sequences for amplification and/or sequencing methods, quality control elements (i.e. such as Key elements or other type of quality control element), unique identifiers (also referred to as a multiplex identifier or “MID”) that encode various associations such as with a sample of origin or patient, or other functional element.

For example, some embodiments of the described invention comprise associating one or more embodiments of an MID element having a known and identifiable sequence composition with a sample, and coupling the embodiments of MID element with template nucleic acid molecules from the associated samples. The MID coupled template nucleic acid molecules from a number of different samples are pooled into a single “Multiplexed” sample or composition that can then be efficiently processed to produce sequence data for each MID coupled template nucleic acid molecule. The sequence data for each template nucleic acid is de-convoluted to identify the sequence composition of coupled MID elements and association with sample of origin identified. In the present example, a multiplexed composition may include representatives from about 384 samples, about 96 samples, about 50 samples, about 20 samples, about 16 samples, about 12 samples, about 10 samples, or other number of samples. Each sample may be associated with a different experimental condition, treatment, species, or individual in a research context. Similarly, each sample may be associated with a different tissue, cell, individual, condition, drug or other treatment in a diagnostic context. Those of ordinary skill in the related art will appreciate that the numbers of samples listed above are provided for exemplary purposes and thus should not be considered limiting.

In preferred embodiments, the sequence composition of each MID element is easily identifiable and resistant to introduced error from sequencing processes. Some embodiments of MID element comprise a unique sequence composition of nucleic acid species that has minimal sequence similarity to a naturally occurring sequence. Alternatively, embodiments of a MID element may include some degree of sequence similarity to naturally occurring sequence.

Also, in preferred embodiments, the position of each MID element is known relative to some feature of the template nucleic acid molecule and/or adaptor elements coupled to the template molecule. Having a known position of each MID is useful for finding the MID element in sequence data and interpretation of the MID sequence composition for possible errors and subsequent association with the sample of origin.

For example, some features useful as anchors for positional relationship to MID elements may include, but are not limited to, the length of the template molecule (i.e. the MID element is known to be so many sequence positions from the 5′ or 3′ end), recognizable sequence markers such as a Key element and/or one or more primer elements positioned adjacent to a MID element. In the present example, the Key and primer elements generally comprise a known sequence composition that typically does not vary from sample to sample in the multiplex composition and may be employed as positional references for searching for the MID element. An analysis algorithm implemented by application 135 may be executed on computer 130 to analyze generated sequence data for each MID coupled template to identify the more easily recognizable Key and/or primer elements, and extrapolate from those positions to identify a sequence region presumed to include the sequence of the MID element. Application 135 may then process the sequence composition of the presumed region and possibly some distance away in the flanking regions to positively identify the MID element and its sequence composition.

Some or all of the described functional elements may be combined into adaptor elements that are coupled to nucleotide sequences in certain processing steps. For example, some embodiments may associate priming sequence elements or regions comprising complementary sequence composition to primer sequences employed for amplification and/or sequencing. Further, the same elements may be employed for what may be referred to as “strand selection” and immobilization of nucleic acid molecules to a solid phase substrate. In some embodiments, two sets of priming sequence regions (hereafter referred to as priming sequence A, and priming sequence B) may be employed for strand selection, where only single strands having one copy of priming sequence A and one copy of priming sequence B is selected and included as the prepared sample. In alternative embodiments, design characteristics of the adaptor elements eliminate the need for strand selection. The same priming sequence regions may be employed in methods for amplification and immobilization where, for instance, priming sequence B may be immobilized upon a solid substrate and amplified products are extended therefrom.

Additional examples of sample processing for fragmentation, strand selection, and addition of functional elements and adaptors are described in U.S. patent application Ser. No. 10/767,894, titled “Method for preparing single-stranded DNA libraries”, filed Jan. 28, 2004; U.S. patent application Ser. No. 12/156,242, titled “System and Method for Identification of Individual Samples from a Multiplex Mixture”, filed May 29, 2008; and U.S. patent application Ser. No. 12/380,139, titled “System and Method for Improved Processing of Nucleic Acids for Production of Sequencable Libraries”, filed Feb. 23, 2009, each of which is hereby incorporated by reference herein in its entirety for all purposes.

Various examples of systems and methods for performing amplification of template nucleic acid molecules to generate populations of substantially identical copies are described. It will be apparent to those of ordinary skill that it is desirable in some embodiments of SBS to generate many copies of each nucleic acid element to generate a stronger signal when one or more nucleotide species is incorporated into each nascent molecule associated with a copy of the template molecule. There are many techniques known in the art for generating copies of nucleic acid molecules such as, for instance, amplification using what are referred to as bacterial vectors, “Rolling Circle” amplification (described in U.S. Pat. Nos. 6,274,320 and 7,211,390, incorporated by reference above) and Polymerase Chain Reaction (PCR) methods, each of the techniques are applicable for use with the presently described invention. One PCR technique that is particularly amenable to high throughput applications include what are referred to as emulsion PCR methods (also referred to as emPCR™ methods).

Typical embodiments of emulsion PCR methods include creating a stable emulsion of two immiscible substances creating aqueous droplets within which reactions may occur. In particular, the aqueous droplets of an emulsion amenable for use in PCR methods may include a first fluid, such as a water based fluid suspended or dispersed as droplets (also referred to as a discontinuous phase) within another fluid, such as a hydrophobic fluid (also referred to as a continuous phase) that typically includes some type of oil. Examples of oil that may be employed include, but are not limited to, mineral oils, silicone based oils, or fluorinated oils.

Further, some emulsion embodiments may employ surfactants that act to stabilize the emulsion, which may be particularly useful for specific processing methods such as PCR. Some embodiments of surfactant may include one or more of a silicone or fluorinated surfactant. For example, one or more non-ionic surfactants may be employed that include, but are not limited to, sorbitan monooleate (also referred to as Span™ 80), polyoxyethylenesorbitsan monooleate (also referred to as Tween™ 80), or in some preferred embodiments, dimethicone copolyol (also referred to as Abil® EM90), polysiloxane, polyalkyl polyether copolymer, polyglycerol esters, poloxamers, and PVP/hexadecane copolymers (also referred to as Unimer U-151), or in more preferred embodiments, a high molecular weight silicone polyether in cyclopentasiloxane (also referred to as DC 5225C available from Dow Corning).

The droplets of an emulsion may also be referred to as compartments, microcapsules, microreactors, microenvironments, or other name commonly used in the related art. The aqueous droplets may range in size depending on the composition of the emulsion components or composition, contents contained therein, and formation technique employed. The described emulsions create the microenvironments within which chemical reactions, such as PCR, may be performed. For example, template nucleic acids and all reagents necessary to perform a desired PCR reaction may be encapsulated and chemically isolated in the droplets of an emulsion. Additional surfactants or other stabilizing agent may be employed in some embodiments to promote additional stability of the droplets as described above. Thermocycling operations typical of PCR methods may be executed using the droplets to amplify an encapsulated nucleic acid template resulting in the generation of a population comprising many substantially identical copies of the template nucleic acid. In some embodiments, the population within the droplet may be referred to as a “clonally isolated”, “compartmentalized”, “sequestered”, “encapsulated”, or “localized” population. Also in the present example, some or all of the described droplets may further encapsulate a solid substrate such as a bead for attachment of template and amplified copies of the template, amplified copies complementary to the template, or combination thereof. Further, the solid substrate may be enabled for attachment of other type of nucleic acids, reagents, labels, or other molecules of interest.

After emulsion breaking and bead recovery, it may also be desirable in typical embodiments to “enrich” for beads having a successfully amplified population of substantially identical copies of a template nucleic acid molecule immobilized thereon. For example, a process for enriching for “DNA positive” beads may include hybridizing a primer species to a region on the free ends of the immobilized amplified copies, typically found in an adaptor sequence, extending the primer using a polymerase mediated extension reaction, and binding the primer to an enrichment substrate such as a magnetic or sepharose bead. A selective condition may be applied to the solution comprising the beads, such as a magnetic field or centrifugation, where the enrichment bead is responsive to the selective condition and is separated from the “DNA negative” beads (i.e. no or few immobilized copies).

Embodiments of an emulsion useful with the presently described invention may include a very high density of droplets or microcapsules enabling the described chemical reactions to be performed in a massively parallel way. Additional examples of emulsions employed for amplification and their uses for sequencing applications are described in U.S. Pat. Nos. 7,638,276; 7,622,280; 7,842,457; 7,927,797; and 8,012,690 and U.S. patent application Ser. No. 13/033,240, each of which is hereby incorporated by reference herein in its entirety for all purposes.

Also embodiments sometimes referred to as Ultra-Deep Sequencing, generate target specific amplicons for sequencing may be employed with the presently described invention that include using sets of specific nucleic acid primers to amplify a selected target region or regions from a sample comprising the target nucleic acid. Further, the sample may include a population of nucleic acid molecules that are known or suspected to contain sequence variants comprising sequence composition associated with a research or diagnostic utility where the primers may be employed to amplify and provide insight into the distribution of sequence variants in the sample. For example, a method for identifying a sequence variant by specific amplification and sequencing of multiple alleles in a nucleic acid sample may be performed. The nucleic acid is first subjected to amplification by a pair of PCR primers designed to amplify a region surrounding the region of interest or segment common to the nucleic acid population. Each of the products of the PCR reaction (first amplicons) is subsequently further amplified individually in separate reaction vessels such as an emulsion based vessel described above. The resulting amplicons (referred to herein as second amplicons), each derived from one member of the first population of amplicons, are sequenced and the collection of sequences are used to determine an allelic frequency of one or more variants present. Importantly, the method does not require previous knowledge of the variants present and can typically identify variants present at <1% frequency in the population of nucleic acid molecules.

Some advantages of the described target specific amplification and sequencing methods include a higher level of sensitivity than previously achieved and are particularly useful for strategies comprising mixed populations of template nucleic acid molecules.

Further, embodiments that employ high throughput sequencing instrumentation, such as for instance embodiments that employ what is referred to as a PicoTiterPlate® array (also sometimes referred to as a PTP™ plate or array) of wells provided by 454 Life Sciences Corporation, the described methods can be employed to generate sequence composition for over 100,000, over 300,000, over 500,000, or over 1,000,000 nucleic acid regions per run or experiment and may depend, at least in part, on user preferences such as lane configurations enabled by the use of gaskets, etc. Also, the described methods provide a sensitivity of detection of low abundance alleles which may represent 1% or less of the allelic variants present in a sample. Another advantage of the methods includes generating data comprising the sequence of the analyzed region. Importantly, it is not necessary to have prior knowledge of the sequence of the locus being analyzed.

Additional examples of target specific amplicons for sequencing are described in U.S. patent application Ser. No. 11/104,781, titled “Methods for determining sequence variants using ultra-deep sequencing”, filed Apr. 12, 2005; PCT Patent Application Ser. No. US 2008/003424, titled “System and Method for Detection of HIV Drug Resistant Variants”, filed Mar. 14, 2008; and U.S. Pat. No. 7,888,034, titled “System and Method for Detection of HIV Tropism Variants”, filed Jun. 17, 2009, each of which is hereby incorporated by reference herein in its entirety for all purposes.

Further, embodiments of sequencing may include Sanger type techniques, techniques generally referred to as Sequencing by Hybridization (SBH), Sequencing by Ligation (SBL), or Sequencing by Incorporation (SBI) techniques. The sequencing techniques may also include what are referred to as polony sequencing techniques; nanopore, waveguide and other single molecule detection techniques; or reversible terminator techniques. As described above, a preferred technique may include Sequencing by Synthesis methods. For example, some SBS embodiments sequence populations of substantially identical copies of a nucleic acid template and typically employ one or more oligonucleotide primers designed to anneal to a predetermined, complementary position of the sample template molecule or one or more adaptors attached to the template molecule. The primer/template complex is presented with a nucleotide species in the presence of a nucleic acid polymerase enzyme. If the nucleotide species is complementary to the nucleic acid species corresponding to a sequence position on the sample template molecule that is directly adjacent to the 3′ end of the oligonucleotide primer, then the polymerase will extend the primer with the nucleotide species. Alternatively, in some embodiments the primer/template complex is presented with a plurality of nucleotide species of interest (typically A, G, C, and T) at once, and the nucleotide species that is complementary at the corresponding sequence position on the sample template molecule directly adjacent to the 3′ end of the oligonucleotide primer is incorporated. In either of the described embodiments, the nucleotide species may be chemically blocked (such as at the 3′-O position) to prevent further extension, and need to be deblocked prior to the next round of synthesis. It will also be appreciated that the process of adding a nucleotide species to the end of a nascent molecule is substantially the same as that described above for addition to the end of a primer.

As described above, incorporation of the nucleotide species can be detected by a variety of methods known in the art, e.g. by detecting the release of pyrophosphate (PPi) using an enzymatic reaction process to produce light or via detection the release of H⁺ and measurement of pH change (examples described in U.S. Pat. Nos. 6,210,891; 6,258,568; and 6,828,100, each of which is hereby incorporated by reference herein in its entirety for all purposes), or via detectable labels bound to the nucleotides. Some examples of detectable labels include, but are not limited to, mass tags and fluorescent or chemiluminescent labels. In typical embodiments, unincorporated nucleotides are removed, for example by washing. Further, in some embodiments, the unincorporated nucleotides may be subjected to enzymatic degradation such as, for instance, degradation using the apyrase or pyrophosphatase enzymes as described in U.S. patent application Ser. No. 12/215,455, titled “System and Method for Adaptive Reagent Control in Nucleic Acid Sequencing”, filed Jun. 27, 2008; and Ser. No. 12/322,284, titled “System and Method for Improved Signal Detection in Nucleic Acid Sequencing”, filed Jan. 29, 2009; each of which is hereby incorporated by reference herein in its entirety for all purposes.

In the embodiments where detectable labels are used, they will typically have to be inactivated (e.g. by chemical cleavage or photobleaching) prior to the following cycle of synthesis. The next sequence position in the template/polymerase complex can then be queried with another nucleotide species, or a plurality of nucleotide species of interest, as described above. Repeated cycles of nucleotide addition, extension, signal acquisition, and washing result in a determination of the nucleotide sequence of the template strand. Continuing with the present example, a large number or population of substantially identical template molecules (e.g. 10³, 10⁴, 10⁵, 10⁶ or 10⁷ molecules) are typically analyzed simultaneously in any one sequencing reaction, in order to achieve a signal which is strong enough for reliable detection.

In addition, it may be advantageous in some embodiments to improve the read length capabilities and qualities of a sequencing process by employing what may be referred to as a “paired-end” sequencing strategy. For example, some embodiments of sequencing method have limitations on the total length of molecule from which a high quality and reliable read may be generated. In other words, the total number of sequence positions for a reliable read length may not exceed 25, 50, 100, or 500 bases depending on the sequencing embodiment employed. A paired-end sequencing strategy extends reliable read length by separately sequencing each end of a molecule (sometimes referred to as a “tag” end) that comprise a fragment of an original template nucleic acid molecule at each end joined in the center by a linker sequence. The original positional relationship of the template fragments is known and thus the data from the sequence reads may be re-combined into a single read having a longer high quality read length. Further examples of paired-end sequencing embodiments are described in U.S. Pat. No. 7,601,499, titled “Paired end sequencing”; and in U.S. patent application Ser. No. 12/322,119, titled “Paired end sequencing”, filed Jan. 28, 2009, each of which is hereby incorporated by reference herein in its entirety for all purposes.

Some examples of SBS apparatus may implement some or all of the methods described above and may include one or more of a detection device such as a charge coupled device (i.e., CCD camera) or confocal type architecture for optical detection, Ion-Sensitive Field Effect Transistor (also referred to as “ISFET”) or Chemical-Sensitive Field Effect Transistor (also referred to as “ChemFET”) for architectures for ion or chemical detection, a microfluidics chamber or flow cell, a reaction substrate, and/or a pump and flow valves. Taking the example of pyrophosphate-based sequencing, some embodiments of an apparatus may employ a chemiluminescent detection strategy that produces an inherently low level of background noise.

In some embodiments, the reaction substrate for sequencing may include a planar substrate, such as a slide type substrate, a semiconductor chip comprising well type structures with ISFET detection elements contained therein, or waveguide type reaction substrate that in some embodiments may comprise well type structures. Further, the reaction substrate may include what is referred to as a PTP™ array available from 454 Life Sciences Corporation, as described above, formed from a fiber optic faceplate that is acid-etched to yield hundreds of thousands or more of very small wells each enabled to hold a population of substantially identical template molecules (i.e., some preferred embodiments comprise about 3.3 million wells on a 70×75 mm PTP™ array at a 35 μm well to well pitch). In some embodiments, each population of substantially identical template molecule may be disposed upon a solid substrate, such as a bead, each of which may be disposed in one of said wells. For example, an apparatus may include a reagent delivery element for providing fluid reagents to the PTP plate holders, as well as a CCD type detection device enabled to collect photons of light emitted from each well on the PTP plate. An example of reaction substrates comprising characteristics for improved signal recognition is described in U.S. Pat. No. 7,682,816, titled “THIN-FILM COATED MICROWELL ARRAYS AND METHODS OF MAKING SAME”, filed Aug. 30, 2005, which is hereby incorporated by reference herein in its entirety for all purposes. Further examples of apparatus and methods for performing SBS type sequencing and pyrophosphate sequencing are described in U.S. Pat. Nos. 7,323,305 and 7,575,865, both of which are incorporated by reference above.

In addition, systems and methods may be employed that automate one or more sample preparation processes, such as the emPCR™ process described above. For example, automated systems may be employed to provide an efficient solution for generating an emulsion for emPCR processing, performing PCR Thermocycling operations, and enriching for successfully prepared populations of nucleic acid molecules for sequencing. Examples of automated sample preparation systems are described in U.S. Pat. No. 7,927,797; and U.S. patent application Ser. No. 13/045,210, each of which is hereby incorporated by reference herein in its entirety for all purposes. Also, the systems and methods of the presently described embodiments of the invention may include implementation of some design, analysis, or other operation using a computer readable medium stored for execution on a computer system. For example, several embodiments are described in detail below to process detected signals and/or analyze data generated using SBS systems and methods where the processing and analysis embodiments are implementable on computer systems.

An exemplary embodiment of a computer system for use with the presently described invention may include any type of computer platform such as a workstation, a personal computer, a server, or any other present or future computer. It will, however, be appreciated by one of ordinary skill in the art that the aforementioned computer platforms as described herein are specifically configured to perform the specialized operations of the described invention and are not considered general purpose computers. Computers typically include known components, such as a processor, an operating system, system memory, memory storage devices, input-output controllers, input-output devices, and display devices. It will also be understood by those of ordinary skill in the relevant art that there are many possible configurations and components of a computer and may also include cache memory, a data backup unit, and many other devices.

Display devices may include display devices that provide visual information, this information typically may be logically and/or physically organized as an array of pixels. An interface controller may also be included that may comprise any of a variety of known or future software programs for providing input and output interfaces. For example, interfaces may include what are generally referred to as “Graphical User Interfaces” (often referred to as GUI's) that provides one or more graphical representations to a user. Interfaces are typically enabled to accept user inputs using means of selection or input known to those of ordinary skill in the related art.

In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft Windows Powershell that employs object-oriented type programming architectures such as the Microsoft .NET framework.

Those of ordinary skill in the related art will appreciate that interfaces may include one or more GUI's, CLI's or a combination thereof.

A processor may include a commercially available processor such as a Celeron®, Core™, Pentium®, Xeon®, or Itanmium® processor made by Intel Corporation, a SPARC® processor made by Sun Microsystems, an Athlon™, Sempron™, Phenom™, or Opteron™ processor made by AMD corporation, or it may be one of other processors that are or will become available. Some embodiments of a processor may include what is referred to as Multi-core processor and/or be enabled to employ parallel processing technology in a single or multi-core configuration. For example, a multi-core architecture typically comprises two or more processor “execution cores”. In the present example, each execution core may perform as an independent processor that enables parallel execution of multiple threads. In addition, those of ordinary skill in the related will appreciate that a processor may be configured in what is generally referred to as 32 or 64 bit architectures, or other architectural configurations now known or that may be developed in the future.

A processor typically executes an operating system, which may be, for example, a Windows®-type operating system (such as Windows® XP, Windows Vista®, or Windows® 7) from the Microsoft Corporation; the Mac OS X operating system from Apple Computer Corp. (such as Mac OS X v10.6 “Snow Leopard” operating systems); a Unix® or Linux-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

System memory may include any of a variety of known or future memory storage devices. Examples include any commonly available random access memory (RAM), magnetic medium, such as a resident hard disk or tape, an optical medium such as a read and write compact disc, or other memory storage device. Memory storage devices may include any of a variety of known or future devices, including a compact disk drive, a tape drive, a removable hard disk drive, USB or flash drive, or a diskette drive. Such types of memory storage devices typically read from, and/or write to, a program storage medium (not shown) such as, respectively, a compact disk, magnetic tape, removable hard disk, USB or flash drive, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with memory storage device.

In some embodiments, a computer program product is described comprising a computer usable medium having control logic (computer software program, including program code) stored therein. The control logic, when executed by a processor, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

Input-output controllers could include any of a variety of known devices for accepting and processing information from a user, whether a human or a machine, whether local or remote. Such devices include, for example, modem cards, wireless cards, network interface cards, sound cards, or other types of controllers for any of a variety of known input devices. Output controllers could include controllers for any of a variety of known display devices for presenting information to a user, whether a human or a machine, whether local or remote. In the presently described embodiment, the functional elements of a computer communicate with each other via a system bus. Some embodiments of a computer may communicate with some functional elements using network or other types of remote communications.

As will be evident to those skilled in the relevant art, an instrument control and/or a data processing application, if implemented in software, may be loaded into and executed from system memory and/or a memory storage device. All or portions of the instrument control and/or data processing applications may also reside in a read-only memory or similar device of the memory storage device, such devices not requiring that the instrument control and/or data processing applications first be loaded through input-output controllers. It will be understood by those skilled in the relevant art that the instrument control and/or data processing applications, or portions of it, may be loaded by a processor in a known manner into system memory, or cache memory, or both, as advantageous for execution.

Also, a computer may include one or more library files, experiment data files, and an internet client stored in system memory. For example, experiment data could include data related to one or more experiments or assays such as detected signal values, or other values associated with one or more SBS experiments or processes. Additionally, an internet client may include an application enabled to accesses a remote service on another computer using a network and may for instance comprise what are generally referred to as “Web Browsers”. In the present example, some commonly employed web browsers include Microsoft® Internet Explorer 8 available from Microsoft Corporation, Mozilla Firefox® 3.6 from the Mozilla Corporation, Safari 4 from Apple Computer Corp., Google Chrome from the Google™ Corporation, or other type of web browser currently known in the art or to be developed in the future. Also, in the same or other embodiments an internet client may include, or could be an element of, specialized software applications enabled to access remote information via a network such as a data processing application for biological applications.

A network may include one or more of the many various types of networks well known to those of ordinary skill in the art. For example, a network may include a local or wide area network that employs what is commonly referred to as a TCP/IP protocol suite to communicate. A network may include a network comprising a worldwide system of interconnected computer networks that is commonly referred to as the internet, or could also include various intranet architectures. Those of ordinary skill in the related arts will also appreciate that some users in networked environments may prefer to employ what are generally referred to as “firewalls” (also sometimes referred to as Packet Filters, or Border Protection Devices) to control information traffic to and from hardware and/or software systems. For example, firewalls may comprise hardware or software elements or some combination thereof and are typically designed to enforce security policies put in place by users, such as for instance network administrators, etc.

### b. Embodiments of the Presently Described Invention

As described above, embodiments of the presently described invention relate to methods, reagents and kits for producing a stable compacted structure of a single stranded concatamer of clonally amplified nucleic acid template that is resistant to dissolution in sequencing applications. More specifically, embodiments of the invention include using one or more dendrimer species to promote the formation of stable secondary structure in a single stranded nucleic acid molecule to generate a compacted structure useful for a variety of applications that include various nucleic acid sequencing techniques.

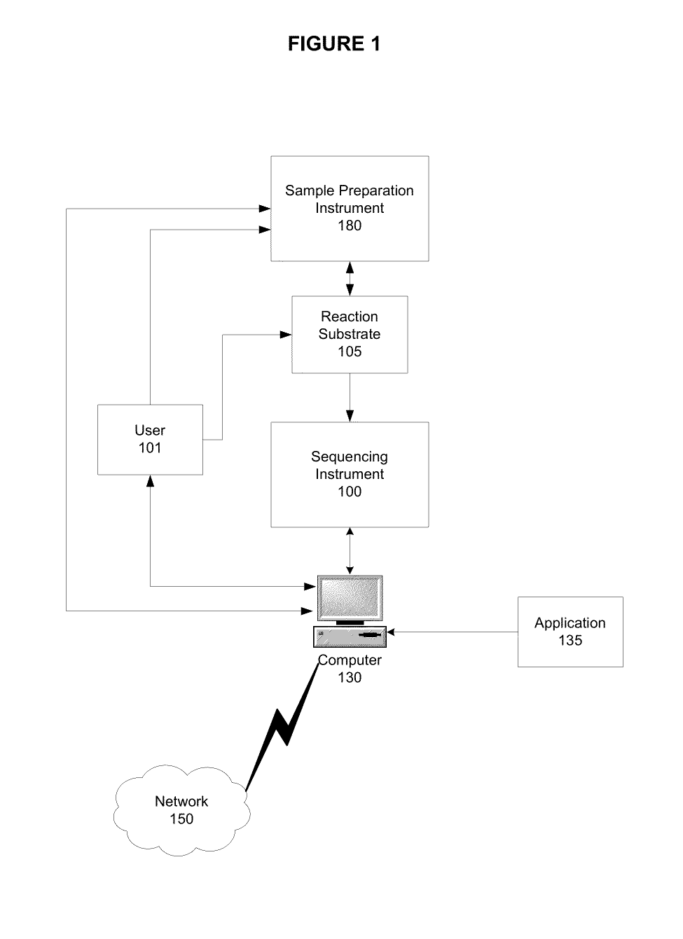

In a typical sequencing embodiment, one or more instrument elements may be employed that automate one or more process steps. For example, embodiments of a sequencing method may be executed using instrumentation to automate and carry out some or all process steps. FIG. 1 provides an illustrative example of sequencing instrument 100 that for sequencing processes requiring capture of optical signals typically comprise an optic subsystem and a fluidic subsystem for execution of sequencing reactions and data capture that occur on reaction substrate 105. It will, however, be appreciated that for sequencing processes requiring other modes of data capture (i.e. pH, temperature, electric current, electrochemical, etc.), a subsystem for the mode of data capture may be employed which are known to those of ordinary skill in the related art. For instance, a sample of template molecules may be loaded onto reaction substrate 105 by user 101 or some automated embodiment, then sequenced in a massively parallel manner using sequencing instrument 100 to produce sequence data representing the sequence composition of each template molecule. Importantly, user 101 may include any type of user of sequencing technologies.

In some embodiments, samples may be optionally prepared for sequencing in a fully automated or partially automated fashion using sample preparation instrument 180 configured to perform some or all of the necessary sample preparation steps for sequencing using instrument 100. Those of ordinary skill in the art will appreciate that sample preparation instrument 180 is provided for the purposes of illustration and may represent one or more instruments each designed to carry out some or all of the steps associated with sample preparation required for a particular sequencing assay. Examples of sample preparation instruments may include robotic platforms such as those available from Hamilton Robotics, Fluidigm Corporation, Beckman Coulter, or Caliper Life Sciences.

Further, as illustrated in FIG. 1, sequencing instrument 100 may be operatively linked to one or more external computer components, such as computer 130 that may, for instance, execute system software or firmware, such as application 135 that may provide instructional control of one or more of the instruments, such as sequencing instrument 100 or sample preparation instrument 180, and/or data analysis functions. Computer 130 may be additionally operatively connected to other computers or servers via network 150 that may enable remote operation of instrument systems and the export of large amounts of data to systems capable of storage and processing. In the present example, sequencing instrument 100 and/or computer 130 may include some or all of the components and characteristics of the embodiments generally described above.

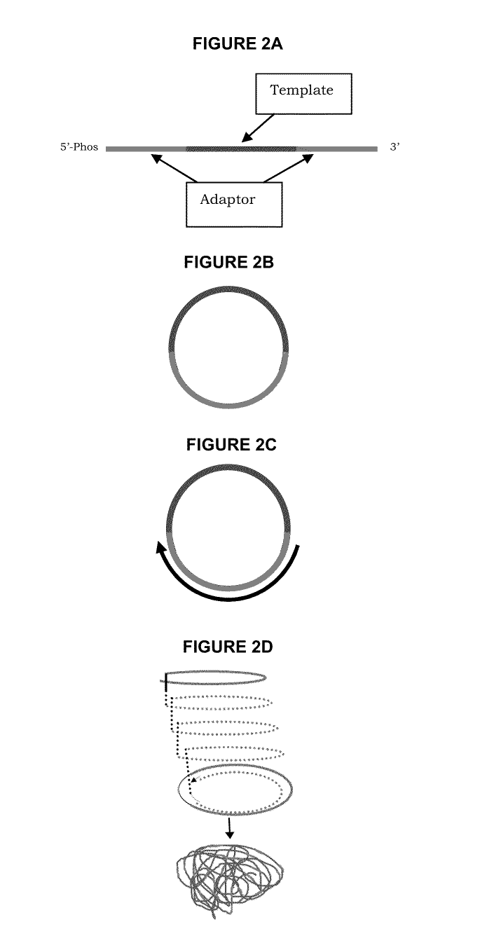

As those of ordinary skill in the art appreciate, what is generally referred to as a DNA “Rolony” or “Nanoball” typically refers to a compacted concatamer molecule that is a result of a clonal, isothermally amplified DNA template molecule. Methods for forming amplified concatamer molecules include what may be referred to as a Rolling Circle Amplification strategy (“RCA”) or Rolling Circle Replication strategy (“RCR”). FIGS. 2A-D provide illustrative representations of a series of steps typically employed for formation of amplified concatamer molecules. For example, FIG. 2A illustrates a step where a linear single stranded DNA (also referred to as “ssDNA”) molecule is adapted with adaptor sequences on both the 3′ and 5′ ends, typically via ligation, with the 5′ end of the adapted nucleic acid molecule further modified to include a phosphate group. However, it will be appreciated that in some embodiments only a single adaptor ligated to either the 3′ or 5′ end. In some embodiments the adaptor sequence(s) may be specially designed to include desirable characteristics that include but are not limited to those that promote directional ligation to the ends of the template nucleic acid (which can either be single stranded or double stranded which can subsequently be melted apart to produce single strands), a resistance to forming what are referred to as adaptor “dimers”, and having ligatable free ends after they are operatively connected to the template molecule.

Next, FIG. 2B illustrates a step of converting the linear adapted ssDNA into a circular ssDNA by joining the 5′ and 3′ ends. In the present example this may be accomplished using the CircLigase ligase enzyme available from Epicentre Biotechnologies (an Illumina company). FIG. 2C further illustrates the step of amplifying the circularized ssDNA and initiating RCA by annealing a forward amplification primer to a complementary sequence in the adaptor region of the circular ssDNA and creating copies using a polymerase with high strand displacement activity such as Phi29 polymerase. Last, FIG. 2D illustrates a step of using isothermal RCA (typically executed at about 30° C.) that produces a linear molecule with a sequence composition comprising concatenated copies of the adapted ssDNA template of FIG. 2A (typically a concatamer of 100's to 1000's of copies of the template sequence). Hydrogen bonds form between regions having amenable sequence composition such as what may be designed into the adaptor region as well as regions of the template sequence that promotes folding of the linear concatamer of amplified copies to form a molecule with a degree of secondary structure that condenses the linear molecule into a three-dimensional structure that is very compact.

It will be appreciated, however, that previous embodiments of nanoballs and rolonies such as those described above have a tendency to unravel and lose compaction when subjected to additional processing steps. It will further be appreciated that the compaction of the previous embodiments rely on Hydrogen bonding (H+ bonds) as described above, which those of ordinary skill understand is a weak interaction. Also, longer molecules typically have increased difficulty with spontaneous folding and the creation of the secondary structure which is due, at least in part, to the randomness of interaction between regions with a propensity to form secondary structure. Environmental conditions such as fluid dynamics, pH levels, and/or certain chemistries (i.e. denaturing chemistries) are generally able to break H+ bonds allowing a compacted molecule to lose secondary structure and linearize. This typically results in a loss of spatial cohesiveness that reduces the advantages and utility of using compacted nucleic acid structures for analysis processes such as sequencing. For example, in embodiments where compacted concatamer molecules are immobilized and closely spaced on a planar substrate, a loss of compaction could result in a “blending” of signals detected from multiple concatamer molecules making it difficult to discriminate which signals emanate from which molecule. In the present example, such signals may appear as a smear over an enlarged area and/or be interpreted as system background or noise signals. Also in the present example the signals may not provide enough detectable signal per unit of area to meet the threshold of detection of a device and be subject to filtering.

Embodiments of the presently described invention provide improvements over the strategy for forming compacted concatamer molecules as illustratively depicted in FIGS. 2A-D. One advantage provided by the described invention includes the replacement of adaptor sequence composition designed to promote secondary structure formation with what those of ordinary skill in the art refer to as branched polyelectrolytes or dendritic macromolecules. It will be appreciated that classes of branched polyelectrolytes or dendritic macromolecules useful in embodiments of the described invention include, but are not limited to, what are generally referred to as dendrons and hyperbranched polymers that comprise positively charged functional groups. Positively charged linear molecules may also work with certain embodiments, such as a linear polyelectrolyte that may for example include polylysine.

One example of a class of branched polyelectrolytes includes what are generally referred to as “dendrimers” that are repeatedly branched, roughly spherical three dimensional molecules with nanometer-scale dimensions. Typical dendrimer species comprise controlled terminal surface chemistry with one or more functional groups that include, but are not limited to, amines, carboxyl, and hydroxyl groups. Species of dendrimer are available as Generation 0 (G0) up to Generation 10 (G10) with each generation having double the number of branches from the previous generation. Hence G0=4 branches, G1=8 and so on.

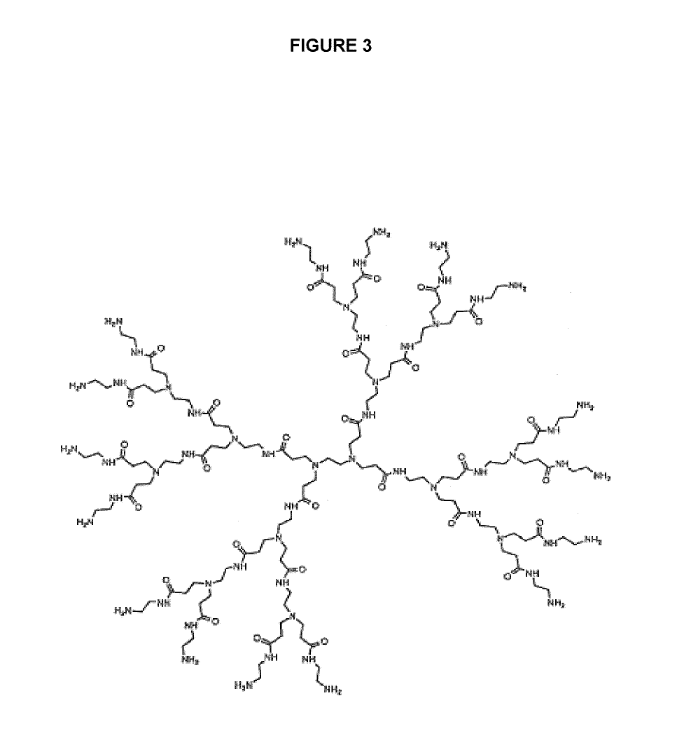

Species of dendrimer useful with the described embodiments includes a branched polyamine that comprises a protonated structure that interacts and forms complexes with the negatively charged backbone of DNA. It will be appreciated that adaptor elements may still be employed with branched polyamines in the embodiments described herein for alternative purposes or to provide improved binding characteristics for the dendrimer species to the nucleic acid. For instance, a Poly(amidoamine) dendrimer species (also referred to as PAMAM) is one of the most well-known dendrimer species, an illustrative example of which is provided in FIG. 3 that illustrates a G2 PAMAM dendrimer molecule with 16 branches having the amine (NH₂) terminal surface chemistry.

It will be appreciated by those of ordinary skill in the related art that PAMAM dendrimer species have been used to compact DNA, for instance for the purpose of injection or transfection of the compacted structure in order to deliver the DNA into cells. The degree of compaction that a particular dendrimer species can provide is dependent, at least in part, on the number and distribution of primary amines found at the terminal ends of the branches of the dendrimer species. The primary amines on the dendrimer species each have a positive charge at pH ranges used during typical DNA polymerase reactions and therefore will bind to the strongly negatively charged phosphate backbone of DNA molecules.

Embodiments of the presently described invention include a method for producing a dendrimer compacted amplified concatamer molecule that exhibits a propensity for folding into tightly compacted structures and exhibit resistance to dissolution in subsequent processing methods, particularly for methods used for nucleic acid sequencing. In the presently described embodiments, one or more species of dendrimers may be added before or during the amplification process to induce folding and secondary structure formation as the concatamer grows with each amplified copy, however high concentrations of dendrimer can have an inhibitory effect on amplification efficiency. Alternatively, one or more species of dendrimers may be added after the amplification process is complete which then induces compaction via formation of secondary structure as the amplified concatamer molecule interacts with and forms bonds to one or more molecules of the dendrimer species. It will be appreciated by those of ordinary skill in the art that some degree of folding may occur in an amplified concatamer molecule due to sequence composition characteristics before the addition or without the aid of the dendrimers. However it is not necessary in the described embodiments that such secondary structure forms.

Those of ordinary skill in the related art will appreciate that certain conditions can have an inhibitory effect on dendrimers ability to form secondary structure which include high pH levels (˜8.5-10 which can de-protonate the dendrimers), and high magnesium (Mg⁺) concentration which compete with the dendrimer species for binding to negatively charged molecules although dendrimer species tend to have better binding efficiency than Mg⁺ due to the existence of more positive charge in localized areas.

It is important to note that one or more dendrimer compacted amplified concatamer molecule's may be immobilized to solid phase substrates for various purposes that include but are not limited to bead substrates (spherical or non-spherical), planar substrates, or cavitated substrates which may include well structures. For example, one or more dendrimer compacted amplified concatamer molecules may be immobilized to bead substrates using techniques known to those of ordinary skill in the related art. The dendrimer compacted amplified concatamer molecule beads may then be used as templates for sequencing methods that include but are not limited to fluorescent detection based methods (i.e. a method for sequencing on a Polonator Instrument), luminescent detection based methods (i.e. detection of the release of pyrophosphate upon nucleotide incorporation that is enzymatically converted to light), or pH detection based methods (i.e. detection of the release of hydrogen ions upon nucleotide incorporation).

Embodiments of dendrimer compacted amplified concatamer molecules may be particularly advantageous for use in embodiments of pH detection based sequencing methods due, at least in part, to characteristics such as conferring substantially no pH buffering in the preferred pH ranges for sequencing that enable faster pH change kinetics (i.e. allowing faster changes in pH levels) than available when using substrates with some pH buffering capacity.

### EXAMPLES

Embodiments of G4 (64 branches with the amine terminal group) and G5 (128 branches with the amine terminal group) PAMAM dendrimer species were used to compact amplified concatamer molecules to form dendrimer compacted amplified concatamer molecules which were then used as the DNA templates in a sequencing by synthesis (SBS) process. In the present example, the SBS process includes a reversible terminator based method that enables detection of signals as single dNTP molecules are incorporated into a growing nucleic acid strand complementary to a template nucleic acid molecule. A terminator dNTP comprising fluorescent label specific to each dNTP species is imaged as each dNTP is incorporated into the nucleic acid strand by a polymerase and then the terminator moiety is cleaved to allow incorporation of the next dNTP molecule. All four reversible terminator bound dNTP species are present during each sequencing cycle.

To determine the optimal dendrimer concentration for dendrimer compacted amplified concatamer generation, the dendrimer species were titrated from 0.1 μM to 100 μM in wash buffer, mixed with amplified concatamer molecules to produce dendrimer compacted amplified concatamer that were deposited on a flow cell and run through 1 cycle of SBS on a Polonator G.007 sequencing instrument (available from Dover Systems). The resulting images were used to qualitatively judge levels of compaction and to select the optimal concentration range of dendrimer species.

Setting Up Dendrimer Compacted Amplified Concatamer Molecules for Sequencing by Synthesis (SBS) on a Polonator Sequencing Instrument:

Amplified concatamer molecules without dendrimers and dendrimer compacted amplified concatamer molecules comprising various dendrimer concentrations were sequenced and imaged on a Polonator G.007 sequencing instrument. To do this the flow cell was prepared according to the Polonator G.007 Operator's Manual and User Guide Rev 1.50, by silinating the 8-lane flow cell with (3-Aminopropyl) triethoxysilane (Sigma-Aldrich cat #440140) that was diluted by adding 60 μl to 1 ml of water. The silinated flow cell was incubated at room temperature for 30 minutes and then flushed with water and air dried. This procedure functionalized the flow cell surface with amine groups. To sequence the amplified concatamer and dendrimer compacted amplified concatamer molecules the following steps were carried out:

Amplified concatamer molecules were diluted in 200 pi of 1× PBS and further diluted at a 1:1 ratio in 1× PBS to a final volume of 10 Sequencing primer with its 5′end modified with an amine group was diluted to 2.5 μM in a 5×SSC buffer containing 0.01% Tween-20 (also referred to as Wash buffer in this protocol). 10 μl of this diluted sequencing primer was added to 10 μl of the diluted amplified concatamer molecules.

Annealing was carried out by heating the amplified concatamer molecules with the sequencing primer at 60° C. for 5 mins, 20° C. for 5 mins followed by a 4° C. pause.

Dendrimer species were diluted in wash buffer and 10 μl of this dilution was added to the primed amplified concatamer molecules to form dendrimer compacted amplified concatamer molecules.

A homobifunctional cross-linker BS3 (Pierce cat #21585) that links the amine groups was prepared by following the Polonator G.007 Operator's Manual and User Guide Rev 1.50, Page #40-41.

20 μl of the diluted BS3 was added to the primed amplified concatamer molecules and 10 μl was added to the dendrimer compacted amplified concatamer molecules and this mixture was loaded on the silinated flow cell and incubated at room temperature for 1 hour.

After incubation of the amplified concatamer and dendrimer compacted amplified concatamer molecules, the flow cell was flushed with about 5 ml of Wash buffer.

SBS was carried out using TruSeq v5 reagents (available from Illumina, Inc.) and that incorporates 1 base per flow of SBS reagents due to its fluorescently labeled reversible terminator chemistry.

The Polonator G.007 sequencing instrument was set to take images of the sequenced Amplified concatamer molecules at different gains.

Ultra High-throughput Test Fragments (UHTFs):

To assess sequencing by synthesis (SBS) using amplified concatamer molecules, 6 test fragments were designed based on the Adenovirus sequence. These test fragments also referred to as Ultra High-throughput Test Fragments (UHTFs) were designed to have varying GC content, homo-polymer stretches and common bases at certain cycle positions, which would be used for cross-talk corrections. The UHTFs are listed below:

UHTF2
(SEQ ID NO: 1)
TACGCAAGCTAAGCCATATCCTTATTAAAGCTTTTTTGAAAGTTAATCGT
UHTF 79
(SEQ ID NO: 2)
ATCGTTCCCCCGCGGCGCCGGGCCCGGCACTAGGCGGGGGCCTAGGTGCA
UHTF 90
(SEQ ID NO: 3)
ACGTAAAAACTTAAAACTCAAACTCAACGCACCAGCCTATGCGCCTGGTC
UHTF 100
(SEQ ID NO: 4)
GCATCCGAGCCCTATCCCCTATCCCCCTATTCTCTTGAGGAGAGTCAACC
UHTF 120
(SEQ ID NO: 5)
GTACGGATAGGGATAGGGGATCGGGGGATAGGCTTAACTCATTTCTCTAC
UHTF 150
(SEQ ID NO: 6)
CGTATTTTTACGTTTTAGATTTAGATTGCGTTATAACCCATACTAGGGGG

Characteristics of these UHTFs are:

The first 4 bases of each UHTF is a single-base T, A, C and G to establish the 1-mer baseline.

UHTF2 has 32% GC contents with a 6-mer of T from cycle 32 to 37, and high AT composition in the beginning of the sequencing. The 6-mer of T will help in evaluating the quality of the sequence after the 1st 30 cycles.

UHTF79, 78% GC contents, very low free energy and thus a stable secondary structure to have a potential “hard-stop” in the sequencing.

UHTF90, 46% GC contents, with 5-mer of A laddering down

UHTF100, 56% GC contents, with ladder of C up to 5-mer

UHTF 120, 50% GC contents, with ladder of G, similar as UHTF 100

UHTF150, 34% GC contents, with ladder of T and 5-mer G in the end. The 5-mer of G will help in evaluating the quality of the sequence after the 1st 30 cycles.

Also, flow cycle position 7, 10, 11, 12, 19, 23, 25, 29, 31, 34, 35, 41, 43, 48 and 50 have three out of the 6 UHTFs with same nucleotides lighting up, which may be used for the cross-talk study

Using UHTFs to Form Amplified Concatamer Molecules:

From the set of six UHTFs four were evaluated for formation of amplified concatamer molecules, which included UHTF 2, UHTF 79, UHTF 120 and UHTF 150. These were chosen based on their varied secondary structures, GC or AT content and homopolymer regions. When these four UHTF samples were tested without dendrimers using a fluorescently labeled detection oligonucleotide, they all appear to form discreet compacted amplified concatamer molecules. However, when exposed to a single cycle of SBS using fluorescently labeled reversible terminators, unraveling is observed in all cases except for UHTF79.

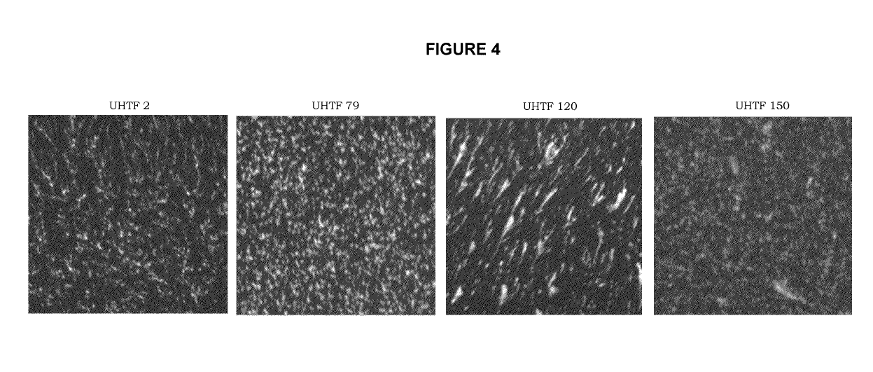

FIG. 4 provides and illustrative example of the effect of a single cycle of SBS on the structure of amplified concatamer molecules. In particular, FIG. 4 demonstrates UHTF 2, UHTF 120 and UHTF 150 amplified concatamer molecules that became unraveled and indistinct after the 1st base incorporation and UHTF 79 amplified concatamer molecules remained intact. It is assumed that the stable secondary structure of UHTF 79 along with the hydrogen bonds formed in the adaptor region helped to maintain the shape withstanding the SBS flows. Even though the same adaptor was part of the other UHTF amplified concatamer molecules, they did not maintain their structure during the SBS flows. Based on these results 1 cycle of SBS that incorporates the first base was established as the standard method to perform quality control (QC) of amplified concatamer molecules.

Compaction of amplified concatamer molecules using G4 and G5 dendrimers: Generation 4 and Generation 5 PAMAM dendrimers were selected to compact amplified concatamer molecules and tested the resulting dendrimer compacted amplified concatamer molecules in sequencing. The procedure to add dendrimer to amplified concatamer molecules includes a dilution of the dendrimer into Wash buffer. A 10 μl aliquot of this dilution is added to the primed amplified concatamer molecules described above followed by the addition of 10 μl of the diluted BS3 so that the total volume is 40 μl.

In the described example, UHTF 120 amplified concatamer molecules with 50% GC content and a ladder of Gs' are unable to maintain their discrete shape after the 1st base is incorporated as demonstrated in FIG. 4 and described above. Hence, the UHTF 120 template was chosen to evaluate the effect of G5 PAMAM dendrimers during and after RCA.

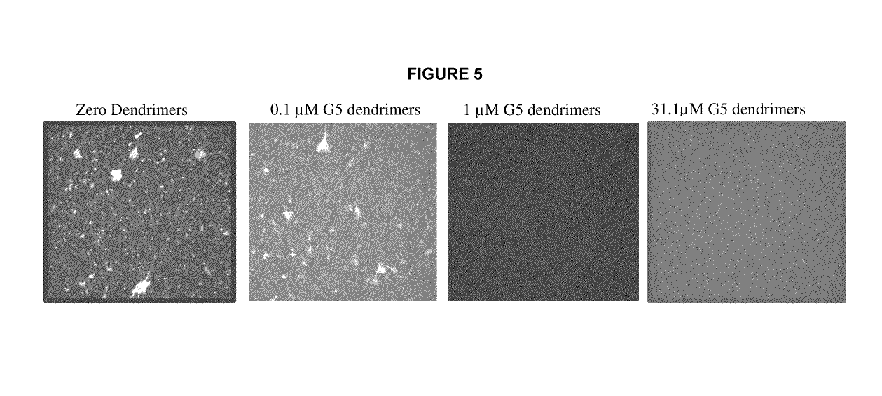

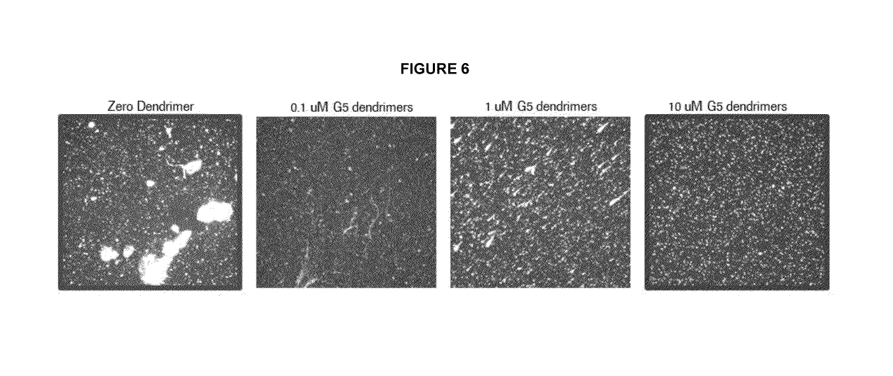

FIG. 5 provides an illustrative example of a Quality Control process by 1 cycle of SBS on UHTF 120 dendrimer compacted amplified concatamer molecules generated using G5 dendrimers during RCA. The results from FIG. 5 suggest that dendrimers have an inhibitory effect on compaction during the RCA reaction especially at higher concentrations (>1 μM) at which point no dendrimer compacted amplified concatamer molecules are formed. In addition, FIG. 6 provides and illustrative example of a Quality Control process by 1 cycle of SBS on UHTF 120 dendrimer compacted amplified concatamer molecules compacted with G5 dendrimers post RCA. The results from FIG. 6 suggest that using higher concentration of dendrimers (˜10 μM) post amplification compacts the dendrimer amplified concatamer molecules better by potentially eliminating the unraveling and clustering effect the SBS flows have on them as seen in the Zero Dendrimer image. It was also determined that the concentration of G5 dendrimers can be increased further without having a detrimental effect.

Evaluating the Effect of Dendrimers on a Mixed Set of UHTF Amplified Concatamer Molecules Over a Series of SBS Flows:

Four sets of UHTF amplified concatamer molecules (UHTF 2, UHTF 79, UHTF 120 and UHTF 150) were compacted with 40 μM G5 dendrimers to form dendrimer compacted amplified concatamer molecules and sequenced by synthesis on the Polonator instrument. The results from this run were compared with a non-dendrimer mixed UHTF amplified concatamer molecules run.

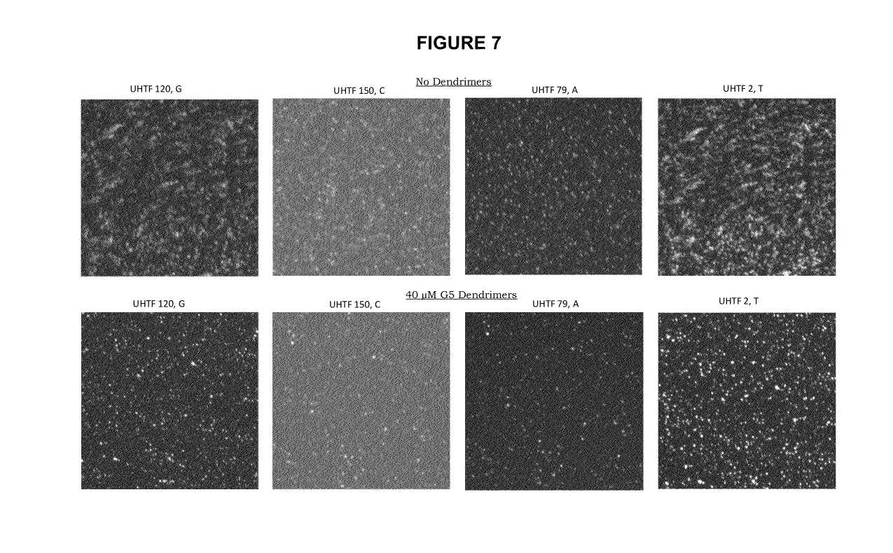

FIG. 7 provides an illustrative example of a comparison of the 1st SBS cycle of SBS between the G5 dendrimer compacted amplified concatamer molecule species and non-dendrimer UHTF amplified concatamer molecules. From the non-dendrimer images, it is evident that UHTF 2, UHTF 120 and to some extent UHTF 150 have started unfolding and unraveling. However, the images from the 40 μM G5 dendrimer run all 4 of the UHTF dendrimer compacted amplified concatamer molecule species remain intact and bright.

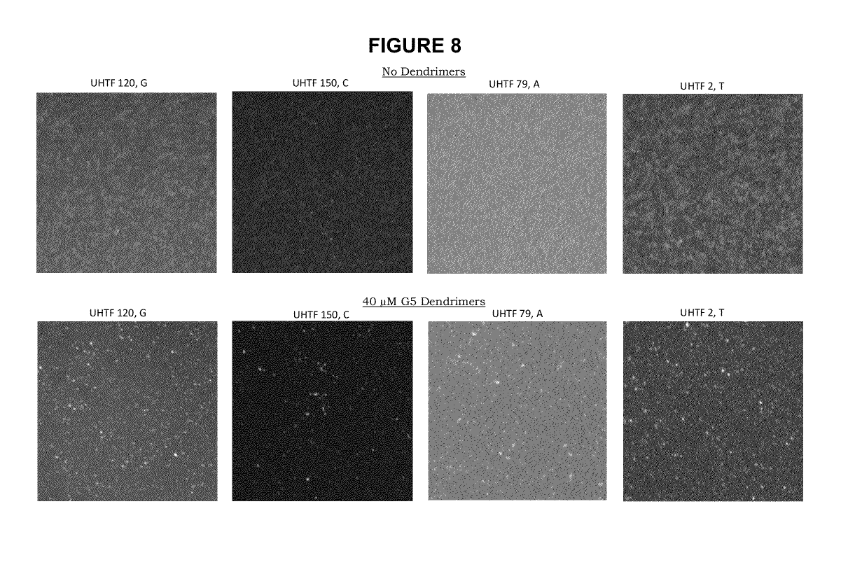

FIG. 8 provides an illustrative example of the comparison of FIG. 7 at SBS cycle 13, following the bases incorporated in UHTF 120, UHTF 150, UHTF 79 and UHTF 2 dendrimer compacted amplified concatamer and non-dendrimer UHTF amplified concatamer molecules. As FIG. 8 illustrates, by the 13th SBS cycle the non-dendrimer UHTF amplified concatamer molecules appear indistinct and dim as compared to the dendrimer compacted amplified concatamer molecules indicating a loss of compaction in the non-dendrimer amplified concatamer molecules.

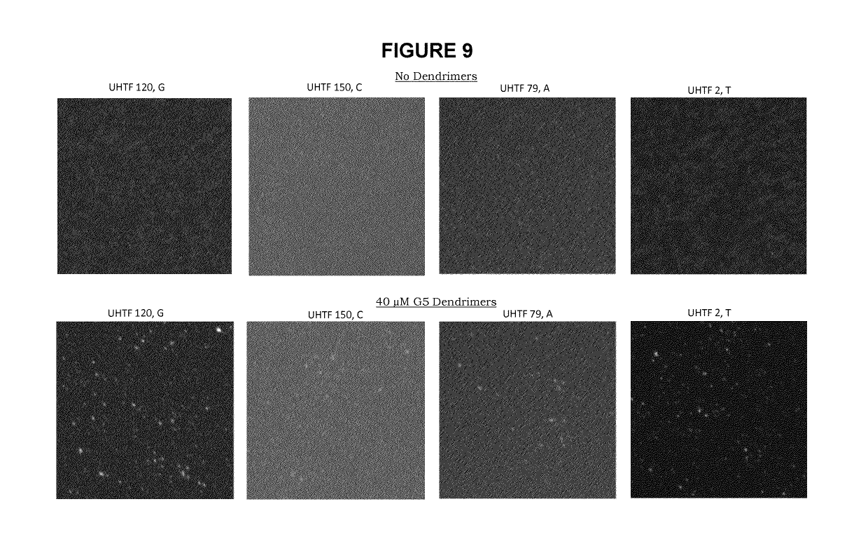

FIG. 9 provides an illustrative example of the comparison of FIG. 7 at SBS cycle 25, following the bases incorporated in UHTF 120, UHTF 150, UHTF 79 and UHTF 2 dendrimer compacted amplified concatamer and non-dendrimer UHTF amplified concatamer molecules. The results from the comparison of FIGS. 7-9 show that over time and with a series of SBS cycles, the amplified concatamer molecules without dendrimers are almost indistinguishable from the background in the image. However, by adding dendrimers, the same amplified concatamer molecule species were able to maintain their shape and withstand the SBS repeated cycles over time.

50-Cycle Sequencing Results Using the Polonator Sequencing Instrument and the Fluorescently Labeled Reversible Terminator Chemistry:

Sequencing performance of UHTF amplified concatamer molecules with and without dendrimers was demonstrated indicating that the presence of dendrimer species does not inhibit sequencing by synthesis processes.

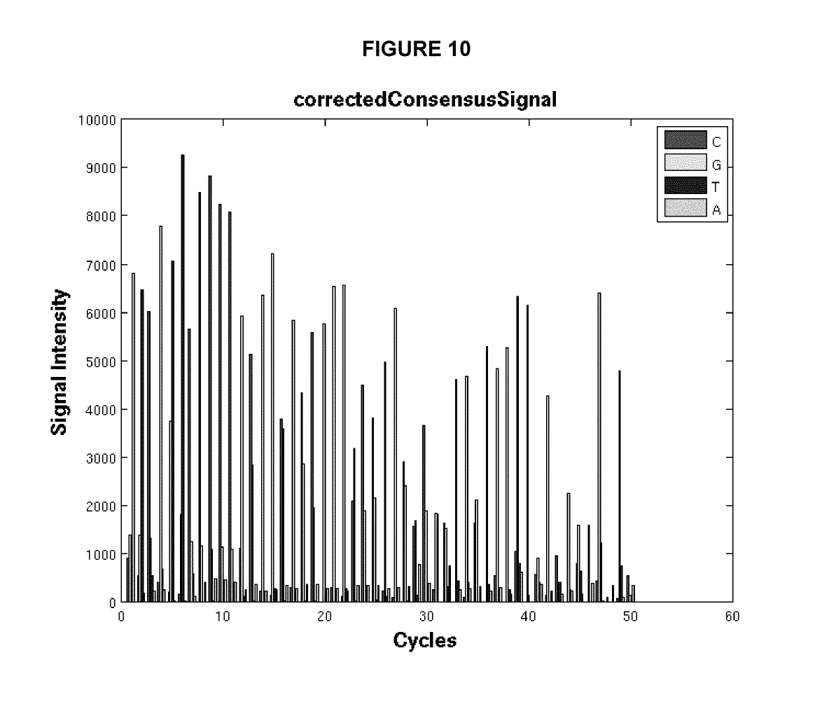

In the context of SBS using amplified concatamer molecules with no dendrimers, UHTF 79 is a 50-mer sequence with 78% GC content that naturally form discretely compacted amplified concatamer molecules that have a resistance to unfolding or unraveling. A 50-cycle sequencing run was done with the UHTF 79 amplified concatamer molecules on the Polonator instrument. FIG. 10 provides and illustrative example of a representation of a phase corrected consensus signal of UHTF 79 amplified concatamer molecules sequenced with no dendrimers demonstrating detected signal intensity values between 6000 and 9000 counts and some moderate signal droop.

TABLE 1

| | 10 bp | | 20 bp | | 30 bp | | 40 bp | | 50 bp | |
|---|---|---|---|---|---|---|---|---|---|---|
| numReads | 5989 | 5989 | 5989 | 5989 | 5989 | 5989 | 5989 | 5989 | 5939 | 5989 |
| zeroErr Reads (%) | 87.7 | 91.4 | 18.9 | 61.5 | 0.03 | 10.9 | 0 | 1.2 | 0 | 0 |
| AvgReadErr (%) | 1.4 | 1.0 | 7.4 | 3.0 | 15.0 | 7.0 | 18.0 | 11.0 | 28.0 | 23.0 |

Table 1 provides read accuracy results before (left side) any correction and after (right side) phase correction on the inverted signal. It is suspected the high error rates seen in the read accuracy demonstrated in Table 1 came from the high GC content and secondary structures present in the UHTF 79 sequence.

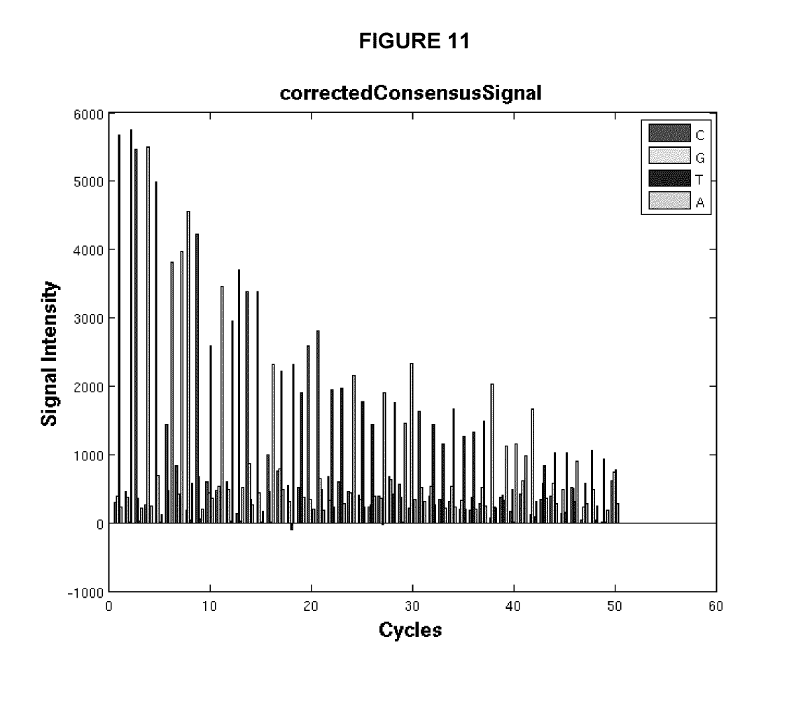

In the context of SBS using amplified concatamer molecules with dendrimers UHTF 2 is a 50-mer sequence with 32% GC content that have a tendency to unravel during the SBS cycles and hence dendrimers help in keeping it condensed. 20 μM G5 dendrimers were used to compact these amplified concatamer molecules and sequenced for 50-cycles on the Polonator instrument. FIG. 11 provides and illustrative example of a representation of a phase corrected consensus signal of UHTF 2 amplified concatamer molecules condensed with 20 μM G5 dendrimers demonstrating detected signal intensity values between 3000 and 6000 counts and some moderate signal droop.

TABLE 2

| | 10 bp | | 20 bp | | 30 bp | | 40 bp | | 50 bp | |
|---|---|---|---|---|---|---|---|---|---|---|
| numReads | 5910 | 5910 | 5910 | 5910 | 5910 | 5910 | 5910 | 5910 | 5910 | 5910 |
| zero Err Reads (%) | 83.5 | 94.3 | 47.1 | 74.7 | 27.1 | 56.2 | 13 | 34.7 | 1.9 | 10.5 |
| AvgReadErr (%) | 2.02 | 0.69 | 5.39 | 2 | 8.5 | 3.2 | 11.64 | 4.4 | 17.8 | 8.6 |

Table 2 provides read accuracy results before (left side) any correction and after (right side) phase correction on the inverted signal. Even though Table 2 demonstrates that the signal values were comparatively lower than the signal values of Table 1, the error rates from the read accuracy table above were also low likely due to reduced GC content. These 50-cycle sequencing runs with and without dendrimers demonstrate that the use of dendrimers to compact the amplified concatamer molecules does not appear to hinder the polymerase's ability to incorporate nucleotides during sequence by synthesis.

Compaction of Amplified Concatamer Molecules Using G6 and G7 Dendrimers at Different Concentrations:

Concentration of G6 and G7 Titrated Dendrimers:

TABLE 3

| | G6 (mM) | G7 (mM) |
|---|---|---|
| 10x | 17.78 | 17.6 |
| 100x | 1.78 | 1.76 |
| 1000x | 0.178 | 0.176 |

UHTF150 Temp1 amplified concatamer molecules were compacted using G6 and G7 dendrimers at 3 different concentrations as provided in Table 3, and imaged using the cleavable Cy3 labeled Sequencing Primer as well as 1-cycle of SBS incorporation. It was observed that the 0.178 mM concentration of G6 dendrimers gave the highest number of bright compacted amplified concatamer molecules, which also had a higher signal for the 1st cycle of SBS incorporation. A similar pattern was observed for G7 dendrimers.

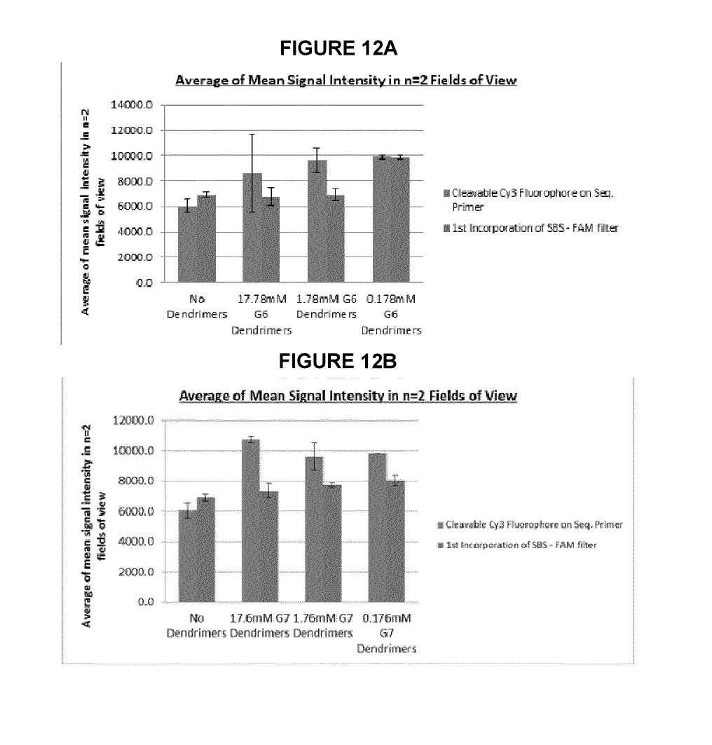

FIGS. 12A and 12B provide illustrative examples that compare the average of mean signal intensity in n=2 fields of view when using G6 and G7 dendrimers respectively.

Single Nucleotide Detection in a Tube Using the ISFET Probe:

In this example the potential of using dendrimer compacted amplified concatamer molecules to detect single nucleotide incorporation using the ISFET based pH probe is discussed. To minimize the Tris content in the amplified concatamer molecules, UHTF 120 amplified concatamer molecules were desalted using the slide-a-lyzer dialysis method in a minimal buffer containing 5 mM MgCl2 and 50 mM KCl. Two conditions were tested, one with G4 PAMAM dendrimers at 100 μM and the other without dendrimers. Positive incorporation was carried out using dATP since the first base on the amplified concatamer molecules was T and for the negative incorporation, dGTP was used. The experiments were performed inside a pressurized Nitrogen glove box to prevent the pH drift due to CO2.

More specifically, amplified concatamer molecules were dialyzed in a minimal buffer containing 5 mM MgCl2 and 50 mM KCl using Slide-A-Lyzer MINI Dialysis device with a MW cut-off of 20 kDa (Pierce cat #69590). These dialyzed amplified concatamer molecules were used to detect the single nucleotide incorporation using the pH probes.

All the steps to detect single nucleotide incorporation are performed inside a pressurized Nitrogen glove box. The procedure includes:

Cleaning the pH Probes twice and calibrating with pH 4 and pH 7 buffers.

Preparing the reagent dilutions and constructing the master mix with 5 mM MgCl2, 50 mM KCl and approximately 10 μM of primed DNA template in a final volume of 90 μl.

Diluting the Bst polymerase to an intermediate concentration of 30 U/μl in a separate tube and adjusting its pH to ˜8.

The master mix was aliquoted into two separate tubes and adding the appropriate dNTP at a final concentration of 100 μM to each tube so that that one will have a positive incorporation and the other sample tube will have no incorporation.

Using a separate probe for each sample tube, adjusting the pH to ˜8.

Recording the pH of the two sample tubes at Time=0 seconds.

Adding 3 μl of the diluted Bst polymerase to the negative incorporation tube and starting the timer.

After 10 seconds, adding the Bst polymerase to the positive incorporation tube and recording the pH for both the tubes at 10 second intervals.

Recording the pH until the reading remains stable in the negative incorporation tube.

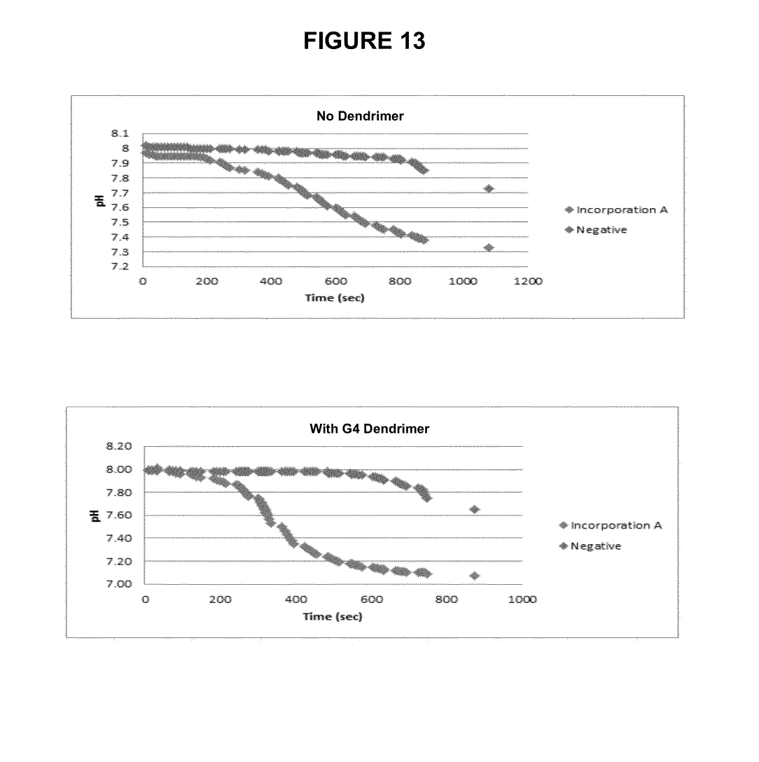

FIG. 13 provides an illustrative example of single nucleotide detection using UHTF 120 amplified concatamer molecules. For instance, the data in the top plot was obtained from amplified concatamer molecules without dendrimers and suggests that the Bst polymerase was able to incorporate the first base releasing protons while the ISFET based pH probe measured the drop in pH starting at the 4 min time point. The pH in the negative incorporation tube remained stable for more than 10 minutes before it started drifting. The bottom plot illustrates single nucleotide detection using UHTF 120 amplified concatamer molecules compacted with 100 μM G4 PAMAM dendrimers which demonstrates a similar pH drop to the non-dendrimer sample during incorporation with Bst. This indicates that the dendrimers at this concentration do not have a measurable negative impact on nucleotide incorporation or the ability to measure pH change.

Having described various embodiments and implementations, it should be apparent to those skilled in the relevant art that the foregoing is illustrative only and not limiting, having been presented by way of example only. Many other schemes for distributing functions among the various functional elements of the illustrated embodiment are possible. The functions of any element may be carried out in various ways in alternative embodiments.

What is claimed is:

1. A method for sequencing a nucleic acid, comprising the steps of:
coupling an adaptor to at least on end of a template nucleic acid molecule;
circularizing the adaptor coupled nucleic acid molecule;
amplifying the adaptor coupled nucleic acid molecule to form a linear amplified concatamer molecule comprising a plurality of copies of the template nucleic acid molecule;
compacting the linear amplified concatamer molecule with a branched polyelectrolyte species to form a branched polyelectrolyte compacted amplified concatamer molecule; and
sequencing the branched polyelectrolyte compacted amplified concatamer molecule to produce a sequence composition of the template nucleic acid molecule.

2. The method of claim 1, wherein:
the branched polyelectrolyte species comprises positively charged functional groups.

3. The method of claim 1, wherein:
the branched polyelectrolyte species comprises a dendrimer species.

4. The method of claim 3, wherein:
the dendrimer species comprises a Poly(amidoamine) dendrimer species.

5. The method of claim 3, wherein:
the dendrimer species is selected from the group consisting of a G4 dendrimer species, a G5 dendrimer species, a G6 dendrimer species, and a G7 dendrimer species.

6. The method of claim 1, wherein:
the branched polyelectrolyte compacted amplified concatamer molecule is resistant to dissolution during the sequencing step.

7. The method of claim 1, wherein:
the branched polyelectrolyte compacted amplified concatamer molecule is operatively coupled to a solid phase substrate.

8. The method of claim 7, wherein:
the solid phase substrate comprises a bead substrate.

9. The method of claim 7, wherein:
the solid phase substrate comprises a planar substrate.

10. The method of claim 7, wherein:
the solid phase substrate comprises one or more reaction wells.

11. The method of claim 1, wherein:
the step of sequencing comprises a sequencing by synthesis step.

12. The method of claim 1, wherein:
the step of sequencing comprises a pyrosequencing step.

13. The method of claim 1, wherein:
the step of sequencing comprises a pH detection sequencing step.

14. The method of claim 1, wherein:
the step of sequencing comprises a sequencing by ligation step.

15. A nucleic acid molecule, comprising:
a compacted nucleic acid concatamer comprising a plurality of copies of a template nucleic acid sequence, a plurality of copies of an adaptor sequence, and one or more branched polyelectrolyte molecules operatively coupled to a backbone of the compacted nucleic acid concatamer.

16. The method of claim 15, wherein:
the branched polyelectrolyte molecules comprise positively charged functional groups.

17. The nucleic acid molecule of claim 15, wherein:
the branched polyelectrolyte species comprises a dendrimer species.

18. The nucleic acid molecule of claim 17, wherein:
the dendrimer molecules comprises a Poly(amidoamine) dendrimer species.

19. The nucleic acid molecule of claim 17, wherein:
the dendrimer species is selected from the group consisting of a G4 dendrimer species, a G5 dendrimer species, a G6 dendrimer species, and a G7 dendrimer species.

20. The nucleic acid molecule of claim 15, wherein:
the compacted nucleic acid concatamer is operatively coupled to a solid phase substrate.

21. The nucleic acid molecule of claim 20, wherein:
the solid phase substrate comprises a bead substrate.

22. The nucleic acid molecule of claim 20, wherein:
the solid phase substrate comprises a planar substrate.

23. The nucleic acid molecule of claim 20, wherein:
the solid phase substrate comprises one or more reaction wells.

24. A method for sequencing a nucleic acid, comprising the steps of:
performing rolling circle amplification of a nucleic acid template in the presence of a branched polyelectrolyte species to form a branched polyelectrolyte compacted amplified concatamer of the nucleic acid template; and
sequencing the branched polyelectrolyte compacted amplified concatamer molecule to produce a sequence composition of the nucleic acid template.
